(19) United States
(12) Patent Application Publication
(10) Pub. No.: US 2009/0175843 A1
(43) Pub. Date: Jul. 9, 2009

(54) Composition for prevention or treatment of urinary tract infection

(75) Inventor: Arnold M. Gans, Englewood, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Medical Nutrition USA, Inc., Englewood, NJ (US)

(21) Appl. No.: 12/317,905

(22) Filed: Dec. 30, 2008

Related U.S. Application Data
(60) Provisional application No. 61/010,381, filed on Jan. 8, 2008.

(51) Int. Cl. A61K 38/48; A61K 36/45; A61P 31/04

(52) U.S. Cl. 424/94.65; 424/732

(57) ABSTRACT

Compositions particularly suitable for preventing or treating urinary tract infections in mammals, especially in humans, comprising a combination of components, the composition comprising an effective amount of: (A) cranberry concentrate, cranberry extract or both; (B) D-mannose; and at least one component selected from the group consisting of: (C) ascorbic acid; (D) bromelain; and (E) inulin. The compositions are preferably in the form of a liquid concentrate such as a beverage drink.

This application claims the benefit of the filing date of U.S. Provisional Patent Application No. 61/010,381 filed Jan. 8, 2008, the disclosure of which is hereby incorporated herein by reference.

### BACKGROUND OF THE INVENTION

The present invention relates to compositions containing a combination of components including cranberry concentrate, suitable for the prevention and treatment of urinary tract infections.

Urinary tract infections (UTIs), or cystitis, pose a serious health problem affecting millions of people each year and poses a particular problem for the elderly, especially those in nursing homes or receiving long term care. It represents the most frequent bacterial infection of residents in long-term care facilities and antibiotic use in its treatment accounts for almost 9% of the cost of nursing home care. The disorder generally affects the kidneys, the ureters, the bladder, and the urethra. Additionally, UTIs afflict a disproportionate share of women and have a recurrence rate of more than 40%. The literature suggests that more than 80% or as many as 90% of acute UTIs in patients with normal anatomical structure and function are caused by strains of E-coli, 10-20% by coagulase-negative Staphylococcus saprophyticus, and 5% or less by other enterobacteriaceae organisms or enterococci. In rare cases, Candida albicans can cause UTI in diabetic patients and S. saprophyticus is said to be the second most common cause in young sexually active women. For purposes of the present invention, a urinary tract infection is any disorder characterized by the presence of a microbial infection of the urinary tract caused by one or more of the preceding organisms and further including pseudomonas aeruginosa, serratia marcescens, enterococcus faecalis, klebsiella pneumoniae, proteus mirabilis, and others known by the medical community to be implicated in UTIs.

Most UTIs, specifically uncomplicated UTIs, are typically treated with prescription oral antibiotics such as ampicillin, co-trimoxazole, trimethoprim, macrodantin, or cephalexin. More aggressive treatments include ciprofloxacin. Trimethoprim (2,4-diamino-5-(3,4,5-trimethoxybenzyl)pyrimidine) or its salt is a bacteriostatic antibiotic mainly used in the prophylaxis and treatment of urinary tract infections. It belongs to the class of chemotherapeutic agents known as dihydrofolate reductase inhibitors. Trimethoprim was formerly marketed by GlaxoWellcome under trade names including “Proloprim,” “Monotrim” and “Triprim,” which are now also produced by various generic pharmaceutical manufacturers. Trimethoprim has also been used in combination with sulfamethoxazole, a sulfonamide antibiotic. The oral antibiotics generally require a prescription order from an attending physician for administration to the patient.

As a result of the increased use of antibiotics in the food supply as well as the heavily prescribed use of antibiotics, strains of bacteria have emerged that exhibit increased resistance to specific antibiotics. This has been observed in E-coli as well, the most common bacteria in UTIs. Additionally, even with the use of antibiotics, the rate of recurrence of UTIs is high, particularly in long care nursing facilities. For this and other reasons it is desirable to avoid the use of antibiotics to treat UTIs and to that end, it would be desirable to prevent UTIs and/or to treat them using alternative compositions and methods.

It has also generally known that ingestion of cranberry juice and extracts or derivatives thereof can provide healthful benefits. While cranberry juice is a pleasant, palatable beverage, recent evidence also suggests that cranberry juice contains one or more ingredients exhibiting pharmacological activity that impart therapeutic benefits, particularly in cases of urinary tract infections. Thus, doctors often recommend cranberry products to patients suffering from urinary tract infections. It is believed that cranberry juice inhibits the adhesion of bacteria to mammalian cells such as epithelial cells. Such inhibition of bacterial attachment to the epithelial cells including the cells lining the urinary tract is believed to promote the shedding of pathogenic bacteria, thus treating and/or preventing infections.

There is a need for safe and effective methods and compositions, including therapeutic compositions for preventing and treating urinary tract infections. There is a further need to provide methods and compositions for treating and preventing urinary tract infections in the form of compositions or remedies that do not require a doctor's prescription, thus permitting greater access and availability to consumers. Furthermore, there is a need to find alternative means for preventing and treating UTIs that avoids the use of antibiotics. There is a need for implementing such treatment and prevention in a simple, reliable and cost effective manner for greater accessibility to those suffering from urinary tract infection.

### SUMMARY OF THE INVENTION

In one embodiment, the present invention relates generally to a composition and method suitable for preventing or treating urinary tract infections in mammals, particularly humans. In another embodiment, the present invention more specifically relates to a composition comprising a combination of components suitable for such use comprising an effective amount of: (A) cranberry concentrate, cranberry extract or both; (B) D-mannose; and at least one component selected from the group consisting of: (C) ascorbic acid; (D) bromelain; and (E) inulin. A particularly preferred composition includes components (A) through (E). The composition is preferably in the form of a liquid concentrate such as a beverage drink.

The composition of the present invention optionally, but preferably further comprises at least one component selected from the group consisting of water, glycerin, phosphoric acid, sodium benzoate, potassium sorbate, sweetener, natural fruit juice or fruit juice concentrate (including other flavoring agents for possible further taste modification), coloring agent and mixtures thereof.

In still another embodiment of the present invention, there is provided a composition for preventing or treating urinary tract infections in mammals, particularly humans, comprising a combination comprising an effective amount of: (A) cranberry concentrate, cranberry extract or both; (B) D-mannose; and at least one component selected from the group consisting of: (C) ascorbic acid; (D) bromelain; and (E) inulin, in a liquid concentrate form, for example in about one ounce (or about 30 ml), suitable for ingestion, to prevent, reduce or eliminate symptoms associated with the infection. Alternatively, where it is desired to provide increased fluid intake, suitably increased liquid volumes can be used to deliver the compositions of the present invention.

In a further embodiment, the composition of the present invention is provided in a particulate, powdered or solid oral dosage form for delivery in, for example, a capsule or tablet. The frequency of delivery in a solid oral dosage form can be adjusted to accommodate the concentration of the primary and optional components (hereinafter defined) so that an effective amount of each is delivered on a regular, continuing or daily basis.

In another embodiment of the present invention, there is provided a method for preventing or treating urinary tract infections in mammals, including humans, comprising administering a composition of the present invention to the mammal in an effective amount for a time sufficient to prevent, reduce or eliminate symptoms associated with the infection. In a preferred embodiment a composition of the present invention is preferably suitably administered to or ingested by an individual on a regular, continuing or on-going basis. In a particularly preferred embodiment, a composition of the present invention is delivered to an individual and the individual further ingests on a daily basis about 900 ml to about 1750 ml of additional fluids, preferably water.

In a further embodiment of the invention, ingestion of compositions of the present invention particularly by residents of long term care facilities can provide advantages in the quality of life experienced by such residents, including prevention or reduction in urinary tract infections, and a reduction in the number of falls experienced by such residents.

### DETAILED DESCRIPTION OF THE INVENTION

The present invention is generally directed to a compositions and methods for preventing and treating urinary tract infections in mammals, preferably in humans. The compositions and methods include effective amounts of a combination of elements referred to hereinafter as “primary components,” namely cranberry concentrate or extract or both, D-mannose, ascorbic acid, bromelain and inulin, as well as effective or useful amounts of optional, but nevertheless useful components hereinafter described.

One skilled in the art can identify a urinary tract infection (UTI). For example, the diagnostic techniques for a UTI typically include, but are not limited to, palpation over the kidney, urinalysis, urine culture (clean catch), urine culture (catheterized specimen), blood culture, intravenous pyelogram scan, computed tomography scan, voiding cystourethrogram, renal ultrasound, renal scan, and renal biopsy.

Symptoms of urinary tract infections are readily identifiable by those skilled in the art, and also by lay consumers. For example, the symptoms of UTI include, but are not limited to, pressure in the lower pelvis, painful urination (dysuria), frequent need to urinate, urgent need to urinate, need to urinate at night, cloudy urine, blood in the urine (hematuria), and foul or strong urine odor. Fever may or may not be a feature at presentation.

An “effective amount” of each component of the composition of the present invention or a mixture of the compositional components including the “primary components,” as hereinafter described and the optional components, also as hereinafter described, is an amount, taken or delivered to an individual, that is effective to prevent and/or treat a urinary tract infection in mammals, including humans, without undue adverse effects such as toxicity, irritation or allergic response, commensurate with a reasonable benefit/risk ratio when used in the manner of the present invention. Such benefit may not necessarily be observed immediately after ingestion, but may be observed after a reasonable period of time after use, including continuing use for a reasonable period of time in order to observe such benefit using standard test methods relating to UTIs. In a preferred form of administration, the primary components of the composition of the present invention are administered “concurrently,” meaning that they are administered at substantially the same time or within a time period selected from 4, 8, 12, 16 and 24 hours of each other; preferably they are administered conjointly. The specific “effective amount” can vary with such factors including, for example: the infectious agent(s) present in particular urinary tract infection being treated; the condition, stage or state of the infection; the physical condition of the patient, including whether they are male or female, their age, the presence of other medical conditions, etc.; the intended duration of treatment; the nature of concurrent therapy, if any; the specific dosage form to be used; the nature and content of optional components included in the composition; the solubility or dispersibility of the dosage form particularly if a solid form is used; the potency of the cranberry concentrate or extract; and the particular dosing regimen. These factors are normally within the skill of the professional who is responsible for administering the composition or applying the method of the present invention, e.g., a medical professional including a doctor or a nurse, a nutritionist, a pharmacist, etc.

The present invention more specifically includes the use of a fruit concentrate, preferably a solid, e.g., powdered, or most preferably, a liquid concentrate obtained from the berries of a plant belonging to the genus Vaccinium, referred hereinafter as “cranberry concentrate,” in combination with other components in order to prepare the composition useful for preventing and treating urinary tract infections in mammals, including humans. The combination of components of the present invention will have complementary activity to enhance the effectiveness of the individual components in the prevention and treatment of such urinary tract infections, including treatment for the relief of symptoms associated with UTIS.

The cranberry component useful in the present invention can be in the form of a concentrate or an extract, in each case either in liquid form or in solid, e.g., powdered or particulate form. For purposes of the present invention a concentrate refers to a product obtained from the whole cranberry source, including the fruit, leaves, etc., typically crushed or macerated to produce a liquid and from which a portion of the water originally present is removed, thereby forming a concentrate. If the water is substantially or wholly removed, the resulting material becomes more highly concentrated, eventually resulting in a solid, for example, powdered, product that includes substantially all of the components originally present in the liquid. A concentrate produced in this manner can be referred to as about a 4:1 concentrate, or about a 5:1, 6:1, 7:1, 8:1, 9:1, or 10:1 concentrate, any one of which may be useful in the present invention as a component of the composition mixture comprising the other components hereinafter described. A 4:1 concentrate is understood to mean that the original liquid has been dehydrated to the extent that about 75% of the water has been removed, for example using methods well known in the art including freeze drying, heating, heating with application of vacuum, etc. Consequently, the concentration of active ingredients in the resulting concentrate product is increased in proportion to the water that is removed; in other words a 4:1 concentrate has about 4 times the concentration, for example on a weight to volume basis, of proanthocyanidins, tannins, vitamins, minerals, etc., compared to their concentration in the original liquid. Cranberry concentrates are useful in the present invention.

In contrast, a cranberry extract refers to a product, either liquid or solid, e.g., powdered or particulate, resulting from a process designed to extract a specific material or materials from the original plant or plant components, either in their original form, or from a liquid resulting from crushing or maceration. Processes for extraction of components are described in the literature, including U.S. Pat. No. 5,912,363, which discloses a method for extracting proanthocyanidins, including oligomeric proanthocyanidins from grape plants containing such compounds, although the general method is applicable to cranberries. Cranberry extraction methods are also described, see, e.g., U.S. Pat. Nos. 5,646,178, 6,608,102, 6,720,353 and other references cited therein. It is noted that the composition of an extract may vary from the original plant material depending on the extraction method used. For example, a method that utilizes polar solvent will tend to extract polar components from the original plant material, which polar components can then be formed as a product, whereas an extraction method that utilizes a non-polar solvent will extract non-polar components. Similarly, a process that utilizes both polar and non-polar solvents, for example sequentially, can extract both types of components for subsequent use. Cranberry extract products are also available commercially as liquids and solids, including powders. Cranberry extracts are useful in the present invention.

Active components in cranberry concentrate are believed to induce an anti-adhesion effects on pathogenic bacteria suspected of causing urinary tract infection. The extract is generally prepared from plant material and juice from berries of plant species of the genus Vaccinium including, but not limited to, Vaccinium macrocarpon (also known as the North American cranberry), Vaccinium microcarpum and Vaccinium oxycoccus. Useful cranberry concentrate may also be in the form of cranberry fruit concentrates, cranberry juice concentrate, cranberry juice powder, cranberry powder, and the like.

Although the ingestion of cranberry juice in moderately large amounts on a daily basis (for example as much as 8 eight ounce glasses twice a day) has been observed to be beneficial in preventing or reducing the effects of UTIs, cranberry juice is high in sugar and high intake can induce weight gain or otherwise cause problems, particularly for diabetic individuals. The use of a concentrate, modified concentrate with reduced sugar or a cranberry extract (containing active components and reduced sugar) can mediate or obviate such effects.

The combination of cranberry concentrate or cranberry extract and the other preferred components of the compositions of the present invention, the primary components, enhance the prevention or treatment efficacy against urinary tract infections with improved therapeutic activity as compared to cranberry concentrate or extract, or even a mixture of concentrate and extract, administered alone. The composition of the present invention can be readily formulated for general use directly by individuals, but is further enhanced when used with the advice and supervision of a health professional, although none of the components requires a prescription. In other words, compositions of the present invention can be dispensed and administered “over the counter.” The term “over the counter” typically means that the composition is determined in accordance with the provisions of the U.S. Food and Drug Administration to be used primarily to prevent or treat a condition that does not require the direct supervision of a physician and is reasonably safe and well-tolerated with little potential for abuse.

Cranberries are a group of evergreen dwarf shrubs in the genus Vaccinium. There are various species of cranberry including Vaccinium oxycoccus, Vaccinium macrocarpon (also known as the North American cranberry), Vaccinium microcarpum, and Vaccinium erythrocarpum. The cranberry concentrate is obtained from plant material such as the fruit, leaves and berries, preferably the berries, from the plant species of the genus Vaccinium, which is believed to contain active ingredients (including, for example, proanthocyanidins, tannins, etc.) that change the shape and/or inhibit the adherence of certain bacterial species to various substrates. The concentrate can be in liquid form that can be orally administered to the patient as a component of a liquid concentrate mixture as hereinafter described. The cranberry concentrate of the present invention is preferably a 4:1 concentrate prepared from the juice of cranberries which have been dehydrated to the point that about 75% of the water originally present in the cranberry juice has been removed. This provides a concentrate with about four times the total amount of active principles, e.g., proanthocyanidins, tannins, vitamins, minerals, etc., compared to the concentration of such components present in the original cranberry juice. A typical cranberry concentrate useful in the present invention comprises about 49 g to about 50 g of water per 100 g of a 4:1 concentrate. Useful concentrates are selected from the group consisting of about 3.0:1 to about 5.0:1; about 3.1:1 to about 4.9:1; about 3.2:1 to about 4.8:1; about 3.3:1 to about 4.7:1; about 3.4:1 to about 4.6:1; about 3.5:1 to about 4.5:1; about 3.6:1 to about 4.4:1; about 3.7:1 to about 4.3:1; about 3.8:1 to about 4.2:1; and about 3.9:1 to about 4.1:1; a cranberry concentrate of about 4:1 is particularly preferred. A suitable commercial 4:1 concentrate is produced by Ocean Spray Cranberries Ingredient Technology Group, Middleboro, Mass. Alternatively, other concentrate ratios are also useful, including about 3.5:1; about 3.7:1; about 3.9:1; about 4.1:1; and the like, as compositions of the present invention are not critically limited to a ratio of 4:1 for the cranberry concentrate, provided that a cranberry concentrate is present in the composition in an effective amount.

Alternatively, the cranberry concentrate (as well as the other primary and optional components as hereinafter described) can be further dried to produce a solid or powdered form useful for delivery in a solid oral dosage form such as a capsule or tablet. Drying can be accomplished using means well known in the art and including lyophilization or freeze drying as well as thermal drying at a suitably elevated temperature with or without the use of vacuum.

In one embodiment of the present invention, there is provided a composition for preventing or treating urinary tract infections in mammals, including humans, comprising a combination or mixture of “primary components,” namely cranberry concentrate or extract, D-mannose, ascorbic acid, bromelain and inulin, as well as effective or useful amounts of optional, but nevertheless useful components hereinafter described in an effective amount to prevent, reduce or eliminate symptoms associated with the infection, and other optional and pharmaceutically acceptable components as hereinafter described.

In a preferred embodiment, the therapeutic composition is in the form of a liquid oral dosage form or beverage for oral consumption by a mammal. It is noted that liquid delivery forms, including solutions, emulsions and mixtures are preferred over solid forms for oral administration, since the active components present in a liquid may be more readily and/or rapidly absorbed by the body. Liquid delivery vehicles may also be more acceptable to patients, in terms of palatability and ease of administration, e.g., if a patient has difficulty swallowing a pill or capsule. Furthermore, delivery of the composition of the present invention in the form of a liquid concentrate may also facilitate patient compliance.

As noted above, the terms “cranberry concentrate” or “cranberry extract” encompass both wet and dried portions of the plant material of various plant species including members of the genus Vaccinium, preferably the portions that are rich in the active agents such as the leaves or fruit in the natural or processed state. Plant material generally includes leaves, fruit (both mature or ripe fruit, and immature or unripe fruit), flowering parts, stems, seeds, bark and roots, and the like that can be ground, shredded or otherwise macerated and reduced in size for convenient use, and concentrates and extracts thereof, as well as forms commercially available including extracts, powders, capsules, gel caps, tablets, liquid, suspension, and tincture forms. Extracts can include both aqueous and organic solvent extracts, e.g., ethanol. If desired, an extract can be dried and the resulting dried extract employed herein. Cranberry concentrates and extracts can be in the form of products including, for example, cranberry concentrates, cranberry fruits, cranberry fruit concentrates, cranberry juice, cranberry juice concentrate, cranberry juice extract, cranberry juice powder, cranberry powder, cranberry powder extract, and the like. Such products are commercially available from food product vendors such as, for example, Ocean Spray, Inc., MA. Preferred products include liquid and powdered cranberry juice concentrate and liquid and powder cranberry extract. For purposes of the present invention, a reference to “cranberry extract” should be understood to include both extract and concentrate and a reference to “cranberry concentrate” should be understood to include both concentrate and extract, unless the context in which the phrase is used would reasonably lead one to conclude that a specific form was intended, e.g., the concentrate, but not the extract, and vice versa.

The cranberry extract can be in the form of a liquid, or as a powder that can be substantially dissolved in a liquid within a suitable or reasonable period of time. Powdered cranberry concentrate or extract can be processed in a form capable of being reconstituted as related to the mixing of dried fruit, vegetable and/or botanical powder with a liquid such as hot or cold water, or fruit juice, tea or other suitable liquid or in some cases with a semi-liquid or other food ingredients to produce a reconstituted product, particularly in combination with the other primary and optional components of the present invention. The reconstituted product can be a juice beverage, concentrate, sauce, pudding, soft drink, or a puree, and can be derived from natural, commercially available fruits, and/or vegetables and/or botanical plants.

There are known methods of partially or completely dehydrating such plant material and juices to produce solid form products. Typically plant material including fruit juices contain from about 84% to 92% of water. Conventional processes for producing dissolvable solid form products include, but are not limited to, spray drying, vacuum shelf or belt drying, drum drying, foam-mat drying, freeze drying and hydration drying. These processes generally involve the concentration of the juices followed by the dehydration of the concentrate through the application of heat and/or vacuum under controlled conditions.

D-mannose is a naturally occurring simple sugar and is available commercially. It is sometimes used as a sugar substitute. D-mannose is found in pineapple and cranberry juice and is extracted from the larch tree to produce a commercial product (“Waterfall” brand, York, GB). Although related to glucose it is absorbed about eight times slower and when ingested it is not converted to glycogen or stored in the liver. Consequently, it does not interfere with blood sugar regulation, even in persons suffering from diabetes. Instead it passes from the upper gastrointestinal tract to the blood stream and, since the body metabolizes only small amounts, the majority is filtered by the kidneys, passes to the bladder and excreted in the urine. However, D-mannose interferes with the ability of bacteria, and specifically E-coli to adhere to the bladder lining and thus allows such bacteria to be flushed from the body in the urine.

D-mannose is used in the composition and methods of the present invention in an effective amount. Levels of D-mannose useful in the composition, based on a liquid concentrate of about 30 ml (or about one ounce) of a mixture of the combination of the primary and optional components or elements recited above, namely the primary components cranberry concentrate, D-mannose, ascorbic acid, bromelain and inulin, as well as useful or effective amounts of optional components discussed hereinafter, include about 100 mg to about 1000 mg D-mannose. Other useful amounts of D-mannose determined on the same basis can be selected from the group consisting of about 125 mg to about 900 mg; about 150 mg to about 800 mg; about 175 mg to about 700 mg; about 200 mg to about 600 mg; about 225 mg to about 500 mg; about 250 mg to about 400 mg; about 200 mg to about 300 mg; and about 175 mg to about 275 mg. A specific amount useful in the present invention can be selected from the group consisting of about 100 mg; about 150 mg; about 175 mg; about 200 mg; about 225 mg; about 250 mg; about 275 mg; about 300 mg; about 325 mg; and about 350 mg. It is particularly preferred that D-mannose be incorporated in the composition of the present invention such that it is present in a liquid concentrate of about 30 ml (or about one ounce) in an amount of about 250 mg.

Ascorbic acid or vitamin C is considered to stimulate or support the immune system, particularly to assist in fighting infectious agents. It is believed that a high intake of vitamin C may increase the acidity of the urine, which may result in a less suitable environment for the growth of E-coli bacteria. Sufficiently high levels to effect such a change in pH may require as much as 1000 mg of ascorbic acid taken throughout the course of a day on a daily basis. The compositions and methods of the present invention preferably include an effective amount of ascorbic acid as a primary component. Levels of ascorbic acid useful in the composition, based on a liquid concentrate of about 30 ml (or about one ounce) of a mixture of the combination of components or elements recited above, namely the primary components cranberry concentrate or extract, D-mannose, ascorbic acid, bromelain and inulin, as well as useful or effective amounts of optional components, include about 50 mg to about 1000 mg of ascorbic acid. Other useful amounts of ascorbic acid determined on the same basis can be selected from the group consisting of about 75 mg to about 950 mg; about 100 mg to about 900 mg; about 125 mg to about 850 mg; about 150 mg to about 800 mg; about 175 mg to about 750 mg; about 200 mg to about 700 mg; about 225 mg to about 650 mg; about 250 mg to about 600 mg; about 275 mg to about 550 mg; about 300 mg to about 500 mg; about 325 mg to about 450 mg; and about 350 mg to about 400 mg. Preferably the composition includes about 375 mg. A specific amount useful in the composition of the present invention expressed as above can be selected from the group consisting of about 50 mg; about 90 mg; about 130 mg; about 170 mg; about 210 mg; about 250 mg; about 290 mg; about 320 mg; about 360 mg; about 375 mg; and about 400 mg. It is particularly preferred that ascorbic acid be incorporated in the composition of the present invention such that it is present in a liquid concentrate of about 30 ml (or about one ounce) in an amount of about 375 mg.

Bromelain is an enzyme typically found in pineapples that is capable of breaking down proteins, typically peptide bonds, and therefore it is referred to as a proteolytic enzyme. In its commercially available form, bromelain is not a single substance or compound, but rather a collection of enzymes and other compounds. Typically, it is a mixture of sulfur-containing protein-digesting enzymes, called proteolytic enzymes or proteases and also contains several other substances in smaller quantities, including perioxidase, acid phosphatase, protease inhibitors, and calcium. Bromelain is mainly comprised of cysteine proteases, with smaller amounts of acid phosphatase, peroxidase, amylase and cellulase. Commercial bromelain is usually derived from the stem of the pineapple plant, which can differ compositionally from bromelain derived from the fruit. The activity of bromelain can be expressed using a variety of enzyme units. The Food Chemistry Codex (FCC) officially recognizes the use of milk clotting units (mcu). The use of bromelain has been shown to reduce inflammation and it may also enhance the effect of antibiotics such as amoxicillin, erythromycin, penicillamine, and penicillin. In a limited study of people with urinary tract infections, participants given antibiotics in combination with bromelain and trypsin, another enzyme, were cured of the infection. (See, Mori S., et al., Acta Obstet. Gynaecol. Japan, 1972; 19: 147-53) However, the required use of an antibiotic with bromelain (as well as a second proteolytic enzyme) limits utility of the combination. In contrast, the present invention does not require the use of an antibiotic, including the risk of bacterial adaptation when antibiotics are used frequently, particularly in patients susceptible to UTIs.

Levels of bromelain useful in compositions of the present invention are typically about 500 mcu to about 3000 mcu for each about one ounce or about 30 ml of liquid composition volume of the mixture of primary components or elements recited above, namely cranberry concentrate, D-mannose, ascorbic acid, bromelain and inulin, and optional components of the present invention discussed hereinafter. The abbreviation “mcu” stands for milk clotting units and refers to a measure of proteolytic enzyme's potency. Unlike vitamins and minerals, which are typically measured in a variety of ways including milligrams (mg), micrograms (mcg), or International Units (IU), a measure of the strength of bromelain, which is an enzyme, is how quickly it can dissolve a protein, in this case milk protein, expressed as milk clotting units (mcu). One generally accepted test method is expressed according to International Milk Clotting Units or IMCU using the International Dairy Federation (IDF) standard 157A: 1997. As noted and for purposes of the present invention, the amount of bromelain is expressed in mcu. Preferably bromelain is present at about 500 mcu to about 3000 mcu; more preferably about 750 mcu to about 2750 mcu; most preferably about 1000 mcu to about 2500 mcu; for example about 1250 mcu to about 2250 mcu; alternatively about 1500 mcu to about 2000 mcu; such as 1750 mcu to about 2200 mcu. A suitable concentration of bromelain for use in the present invention is selected from the group consisting of about 1000 mcu; about 1250 mcu; about 1500 mcu; about 1750 mcu; about 2000 mcu; about 2250 mcu; about 2500 mcu; about 2750 mcu; and about 3000 mcu. Although no standard conversion is generally accepted between the activity (in mcu) of a given sample of bromelain and its weight (e.g., in mg), typical values are about 3000 mcu to about 5000 mcu per gram or about 3 mcu to about 5 mcu per mg; for example about: 3.0, 3.1, 3.2, 3.3, 3.4, 3.5, etc., in tenths of a unit to about 5 mcu per mg. Thus, if desired and convenient, an approximate conversion can be made to mg for each of the above values expressed in mcu. By way of example and not limitation, the following conversions can be obtained based on the value selected for mcu/gm (which may be indicative of the activity of a particular sample of bromelain) and the amount of bromelain that may be present in a composition of the present invention:

| | Conversion factor, mcu/mg | | | | |
|---|---|---|---|---|---|
| Amount of | 3 | 3.5 | 4 | 4.5 | 5 |
| bromelain, mcu | Calculated Equivalent Weight, mg | | | | |
| 500 | 166 | 142 | 125 | 111 | 100 |
| 2000 | 667 | 571 | 500 | 444 | 400 |
| 3000 | 1000 | 857 | 750 | 666 | 600 |

For purposes of calculation, a value of 4 mcu/mg can be used to estimate the amount of bromelain in a useful composition of the invention, recognizing that a specific value will depend on the activity of the particular bromelain product used, which, of course can be determined by accepted test methods, as noted above. Thus, in a preferred composition comprising about 2000 mcu bromelain, the equivalent estimated weight would be about 500 mg.

Compositions of the present invention include at least one fructo-oligosaccharide (FOS), also referred to as a prebiotic. Prebiotics generally stimulate the growth and/or activity of beneficial bacteria production in the intestines. FOS can help stimulate different beneficial strains of bacteria found in the intestines or gut of a mammal, particularly a human being. A preferred FOS suitable for use in compositions of the present invention is inulin. It is available commercially (e.g., Orafti Ltd., Tienen, Belgium and the Dutch company Cosun) and is considered a food additive that is generally regarded as safe. Inulin is referred to as a fructan and storage carbohydrate, and belongs to a group of naturally occurring carbohydrates containing non-digestible fructo-oligosaccharides. FOS or inulin are natural carbohydrates found in thousands of plants worldwide, from which it can be extracted, including numerous edible plant species such as chicory root, artichoke, Jerusalem artichoke, leek, onion, asparagus, wheat, barley, rye, garlic, jicama, tomatoes and bananas. Inulin from chicory root is typically obtained from the Mediterranean herb, Cichorium intybus, which has also been naturalized in North America; it is often used as a coffee substitute or supplement. Inulin is considered nutritionally beneficial, for example, as a prebiotic for promotion of good intestinal flora and also as an ingestible carbohydrate or sugar replacement with a similar sweet taste. Inulin is a prebiotic in that it promotes the development of probiotics, which are living microorganisms, which typically require controlled storage conditions or may be unstable in a composition. As a prebiotic, inulin is believed to stimulate the growth and/or activity of friendly and healthy intestinal bacteria which supports good colon health and which is beneficial, in combination with other components of the composition of the present invention, for preventing urinary tract infections. Inulin is suitable for people who are on restricted diets, particularly related to diabetic conditions or sensitivity since inulin has a very low glycemic index; a mildly sweet taste; is not absorbed; and does not affect blood sugar levels. Inulin is metabolized in a manner similar to soluble, dietary fiber and resists digestion in the upper gastrointestinal tract, but is fermented by microflora in the large intestine. For that reason, inulin provides most of the same positive effects of soluble fiber in the diet.

Useful levels of FOS or inulin are typically about 100 mg to about 1500 mg for each about one ounce or about 30 ml of liquid composition volume of the mixture of primary and optional components of the present invention. Preferably inulin is present at about 125 mg to about 1300 mg; more preferably about 150 mg to about 1100 mg; most preferably about 200 mg to about 900 mg; for example about 225 mg to about 700 mg; alternatively about 250 mg to about 500 mg; such as 200 mg to about 400 mg. A suitable concentration of inulin for use in the present invention is selected from the group consisting of about 100 mg; about 125 mg; about 150 mg; about 175 mg; about 200 mg; about 225 mg; about 250 mg; about 275 mg; about 300 mg; about 325 mg; and about 350 mg.

A mixture comprising “primary components,” namely cranberry concentrate or extract, D-mannose, and at least one component selected from the group consisting of ascorbic acid, bromelain and inulin, is effective for preventing and/or treating urinary tract infections. The combination can be formulated into pharmaceutical compositions. Standard pharmaceutical formulation techniques are used, such as those disclosed in Remington's Pharmaceutical Sciences, Mack Publishing Company, Easton, Pa.

In addition to effective amounts of the combination of elements recited above, namely the “primary components” cranberry concentrate and D-mannose, and at least one of ascorbic acid, bromelain and inulin, the compositions of the present invention can further comprise optional, but additionally useful, components in suitable or effective amounts. The phrase “optional components,” as used herein, includes one or more compatible solid or liquid fillers, diluents or encapsulating substances, which are suitable for administration to mammals, particularly humans. Such optional components preferably further comprises at least one component selected from the group consisting of water (preferably pyrogen-free, filtered water), glycerin, phosphoric acid, sodium benzoate, potassium sorbate, sweetener, natural fruit juice or fruit juice concentrate such as apple juice and the like (including other flavoring agents for possible further taste modification), other liquids such as tea, herbal tea and the like, coloring agent and mixtures thereof. The term “pyrogen-free” is generally understood to mean that the water is free or substantially free of microbial debris capable of eliciting an immunogenic fever response in the individual who consumes it. The term “compatible,” as used herein, means that the optional components of the composition are capable of being comingled or mixed with the primary components and with each other in a manner such that there is no interaction that would substantially reduce the efficacy of the composition under conditions of ordinary or suitable use. Such optional components must, of course, be of sufficiently high purity, sufficiently low toxicity and used at a concentration so that they are suitable for administration to mammals, particularly humans.

The types of optional components that may be used can be varied depending on whether the composition of the present invention is in liquid or substantially non-liquid form, e.g., powder or solid mixture. Examples of substances which can be used as optional components include: sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as cottonseed oil, sesame oil, olive oil, peanut oil, and corn oil; polyols such as propylene glycol, glycerine, sorbitol, mannitol, and polyethylene glycol; alginic acid; emulsifiers; wetting agents, such sodium lauryl sulfate; coloring agents; flavoring agents; tableting agents, stabilizers; antioxidants; preservatives; and filtered and/or pyrogen-free water.

Optional components for oral administration include sugars, starches, cellulose and its derivatives, malt, gelatin, talc, calcium sulfate, vegetable oils, synthetic oils, polyols, alginic acid, phosphate buffer solutions, emulsifiers, isotonic saline, fruit juices, teas and pyrogen-free water.

Sweeteners suitable for use in the present invention include sugar and artificial sweeteners. Suitable artificial sweeteners include, for example, Acesulfame potassium is a calorie-free artificial sweetener, also known as “Acesulfame K” or “Ace K,” the letter K being the symbol for potassium, and marketed under various trade names. In the European Union it is also identified under its E number or additive code, E950. Acesulfame K is about 180 to about 200 times sweeter than sucrose or table sugar, as sweet as aspartame, also a suitable sweetener, about half as sweet as saccharin, another suitable sweetener, and about one-quarter the sweetness of sucralose, another suitable sweetener. The preferred sweetener for use in the present invention is an artificial sweetener, more preferably sucralose or acesulfame K, even more preferably a mixture of sucralose and acesulfame K.

Compositions of the present invention can include other sweetening or flavor modifying or enhancing agents including natural fruit juices such as apple juice and the like (especially juices in addition to the cranberry juice concentrate already present in the composition), fruit flavors, flavoring agents, menthol, peppermint, and the like.

The compositions of the present invention can be in any of a variety of forms, suitable, for example, for oral administration. Depending upon the particular route of administration desired, a variety of pharmaceutically acceptable excipients well-known in the art can be used. These include solid or liquid fillers, diluents, hydrotropes, surface-active agents, and encapsulating substances. Optional pharmaceutically active materials can be included, which do not substantially interfere with the functional effects of the composition, including, for example, antimicrobial and anti-adhesion activity. The amount of one or more excipients employed in conjunction with the primary components, namely cranberry concentrate or extract or both, D-mannose, ascorbic acid, bromelain and inulin, as well as effective or useful amounts of optional, but nevertheless useful components herein described is sufficient to provide a practical or suitable quantity of the composition. Those of ordinary skill in the art know techniques and components, including excipients, for preparing dosage forms useful in the methods of the present invention.

The compositions of the present invention typically are prepared by admixing the primary components, namely cranberry concentrate or extract or both, D-mannose, ascorbic acid, bromelain and inulin, as well as effective or useful amounts of optional, but nevertheless useful components as described herein. The mixture can be further formulated with pharmaceutically acceptable excipients admixed into a form suitable for administration to mammals, including humans. The composition will generally be administered orally. Conventional methods are suitable, such as administering the components of the composition, particularly the primary components, in tablets, suspensions, solutions, emulsions, capsules, powders, syrups, and the like.

Various oral dosage forms can be used, including such solid forms as tablets, capsules, granules and bulk powders. Tablets can be compressed, tablet triturates, enteric-coated, sugar-coated, film-coated, or multiple-compressed. Such forms can contain suitable binders, lubricants, diluents, disintegrating agents, coloring agents, flavoring agents, flow-inducing agents, and melting agents. Liquid oral dosage forms include concentrates (in which the amount of liquid is limited to the amount needed to disperse at least the primary components in order to facilitate swallowing of the composition by an individual), aqueous solutions, emulsions, suspensions, solutions and/or suspensions reconstituted from granules, and containing suitable solvents, preservatives, emulsifying agents, suspending agents, diluents, sweeteners, melting agents, coloring agents and flavoring agents.

Oral compositions in the form of liquid solutions, emulsions, suspensions, dispersions, and the like are preferred. Pharmaceutically acceptable components or excipients suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methylcellulose, sodium carboxymethyl cellulose, Avicel® RC-591, tragacanth and sodium alginate; typical wetting agents include lecithin and polysorbate 80 or TweenO 80; and typical preservatives include methylparaben and sodium benzoate. Oral liquid compositions can also contain one or more components such as sweeteners, flavoring agents and colorants disclosed herein. Excipients useful in the present invention are well-known in the art and are generally described, for example, in the “Handbook of Pharmaceutical Excipients,” Fifth Edition, R. C. Rowe et al. editors, Pharmaceutical Press, 2006.

The compositions of the present invention can be compressed by usual methods into solid oral dosage forms such as single or multilayer tablets. Moreover, the preparations can be produced in the form of coated tablets. Additionally, the preparations of the present invention can be provided in the form of hard shell capsules. It is also contemplated that the compositions of the present invention can be formulated into the form of dry powder mixes for preparing beverages containing the active or primary and optional ingredients. In general, the various oral dosage forms of the present compositions are prepared by conventional procedures and techniques known in the art. The applicability of such methods and techniques to the formulation of the compositions of the present invention will be apparent to those skilled in the art.

Pharmaceutically acceptable excipients suitable for preparation of unit dosage forms for oral administration are well known in the art. Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide can be used to improve flow characteristics of a particulate or powder mixture during manufacture of the dosage form. Coloring agents, such as the FD&C dyes, can be added for appearance. Sweeteners and flavoring agents, such as aspartame, saccharin, menthol, peppermint, and fruit flavors, are useful adjuvants, particularly for chewable tablets or for use in taste masking or for improving the palatability of the dosage form. Capsules typically comprise one or more solid diluents disclosed above. The selection of excipients depends on secondary considerations such as taste, cost, and shelf stability, and the like, which are not critical for the purposes of the present invention and such selections can be made by a person skilled in the art.

Solid dosage form compositions of the present invention can also be coated by conventional methods, typically with pH or time-pendent coatings, such that the subject compound is released in the gastrointestinal tract in the region particularly suitable for absorption, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit® coatings, waxes and shellac.

The pH of the compositions of the present invention can be adjusted by addition of a pharmaceutically acceptable acid or base or buffered at a selected pH. Suitable acids include, for example, hydrochloric acid and carboxylic acids such as citric acid, tartaric acid and succinic acid. Suitable bases include, for example, the oxides and hydroxides of calcium, potassium, sodium and magnesium, alkaline quaternary compounds, alkaline amino acids, and mixtures thereof. Desirably, compositions of the present invention include pH adjusting substances that are effective for maintaining the pH of the urine of the individual to whom the composition is administered at a desirably acidic level. Generally, urine pH in a healthy human ranges from about 5.5 to about 7.5 in a bell curve type statistical distribution. In people who are not vegetarians, the pH of urine tends to be acidic, e.g., average for normal human urine is slightly acidic at a pH of less than about 6.0, for example about 5.7±0.2, in other words about 5.5 to about 5.9. The pH of urine of persons who are vegans is also acidic, but tend to be slightly higher, for example about 6.1±0.4 and for persons who are lacto-ovo vegetarians, urine pH is typically about 5.9±0.4. (Ausman, L. M., et al., J. Renal Nutrition, 18, 5, 456, 2008) Conversely, most of the bacteria responsible for urinary tract infections cause urine to be more alkaline, e.g., producing a pH of the urine in the range of about 7 to about 8. A diet rich in citrus fruits, legumes, and vegetables also tends to raise the pH and to produce urine that is more alkaline. Most preferably, compositions of the present invention are adjusted to achieve and/or maintain a slightly acidic pH of the urine, typically, a pH of less than about 7; for example about 6 to about 7.

It is also within the scope of this invention to administer each of the primary components of the mixture individually or in combinations comprising fewer than all. Thus, it is possible to formulate the components into separate or combination dosage forms in accordance with procedures hereinbefore and hereinafter described for the composition utilizing all of the primary and/or optional components.

The compositions of the present invention can be provided in a unit dosage form. As used herein, a “unit dosage form” is an amount of the combination of primary components, namely cranberry concentrate or extract or both, and D-mannose, and at least one component selected from the group consisting of ascorbic acid, bromelain and inulin, which is suitable for administration to mammals, including humans, in a single dose, according to good medical, pharmaceutical or nutritional practice, as appropriate. A preferred composition includes ascorbic acid and a particularly preferred composition includes each of cranberry concentrate or extract or both, D-mannose, ascorbic acid, bromelain and inulin. Such unit dosage forms typically include about 500 mg to about 15,000 mg; alternatively, an amount selected from the group consisting of about 750 mg to about 14,000 mg; about 1000 mg to about 13,000 mg; about 1250 mg to about 12,000 mg; about 1500 mg to about 11,000 mg; about 1750 mg to about 10,000 mg; about 2000 mg to about 9,000 mg; about 2250 mg to about 8,000 mg; and about 775 mg to about 9,500 mg. The weight of a specific unit dosage form can be selected from the group consisting of about 750 mg; about 1000 mg; about 1250 mg; about 1500 mg; about 1750 mg; about 2000 mg; about 2250 mg; about 2500 mg; about 2750 mg; about 3000 mg; about 3250 mg; about 3500 mg; about 3750 mg; about 4000 mg; and about 4250 mg. A particularly useful dosage form comprises about 3375 mg of cranberry concentrate or extract or both, D-mannose, ascorbic acid, bromelain (converted to weight in mg based on the use of about 2000 mcu and a conversion factor of about 4 mcu/mg) and inulin, as well as effective or useful amounts of optional, but nevertheless useful components, for example, including filtered water, sweetener, stabilizer, color, etc., as herein described. The above amounts of the primary components are usefully dispersed in a liquid volume of about 0.5 oz to about 1.5 oz (about 15 ml to about 45 ml); alternatively, about 0.75 oz to about 1.25 oz (about 22.5 ml to about 37.5 ml); for example, about 0.9 oz to about 1.1 oz (about 27 ml to about 33 ml); preferably the composition is present in about 1 oz or about 30 ml of liquid, most of which is typically water, introduced with the cranberry concentrate and/or added as water per se. These volumes of liquid can be considered to deliver the composition of the present invention in a concentrated form, i.e., where the amount of liquid present is sufficient to that useful to disperse at least the primary components and to facilitate swallowing of the resulting composition by an individual.

It is also suitable to deliver the compositions of the present invention with a larger volume of liquid, typically water, such that the individual receives additional fluid which may be useful in the prevention or treatment of a urinary tract condition, especially where “flushing” of the kidneys and/or more frequent emptying of the bladder is desirable. Alternatively, a composition of the present invention, even in its more concentrated form can be diluted with additional water (or fruit juice, tea or other suitable fluid) in order to increase the liquid volume consumed by the individual. Suitable fluid amounts can be determined by a skilled professional, and include, for example, the use of about 1.5 times to about 100 times the volumes suggested above (such as 0.5-1.5 fluid ounces); for example, about 2 times to about 75 times; about 3 times to about 50 times; about 4 times to about 40 times; about 5 times to about 30 times; about 6 times to about 20 times; about 7 times to about 15 times; such as about 4 fluid ounces, about 8 fluid ounces, about 16 fluid ounces, and higher (about 120 ml, 240 ml, about 480 ml, and higher).

Alternatively, it may be desirable to deliver compositions of the present invention as part of a comprehensive fluid delivery regimen such as a component of a daily fluid volume objective of up to about 1750 ml, including, for example, a fluid volume of the present composition or as a component of total fluid intake, of at least about 500 ml or about 750 ml; more preferably at least about 900 ml, 1000 ml, 1250 ml, 1500 ml, 1600 ml, 1650 ml and the like. Increased fluid volume, particularly including compositions of the present invention may be particularly useful in assisting to remove or flush from the body in the urine undesirable materials, including bacteria, from the urinary tract, before, during or after one or more indicators of UTI have been observed. Thus, compositions of the present invention are effectively used in a preferred method in combination with sufficient hydration according to ingestion of daily fluid volumes as described above.

In a further embodiment, the composition of the present invention is provided in a powdered or solid oral dosage form for delivery in, for example, a capsule or tablet. The frequency of delivery in a solid oral dosage form can be adjusted to accommodate the concentration of the primary and optional components (herein described) so that an effective amount of each is delivered on a regular, continuing or daily basis. Dosage forms comprising compositions of the present invention can be prepared by incorporating freeze-dried or lyophilized particulate or powder mixes of the primary and optional components into tablets or capsules. Freeze-drying or lyophilization of components to produce small particle size particulates or powders can facilitate disintegration of the solid dosage form composition by allowing rapid permeation by the aqueous media, promoting timely delivery and dispersion or dissolution of the components of the composition. Suitable methods of freeze-drying are well known in the art and commonly employed. Any suitable conventional method of freeze-drying can be utilized.

Similarly, unit dose forms of the combination can be vacuum dried. Vacuum drying involves at least the partial drying of the compositions at temperatures above compositions' collapse temperature. Freeze drying, on the other hand, involves the drying of composition at temperatures below the composition's collapse temperature. Any suitable method of vacuum drying can be used.

In another embodiment of the present invention, there is provided a method for treating or preventing a urinary tract infections in mammals, including humans, comprising administering a therapeutic or effective composition comprising a combination of the primary components, namely cranberry concentrate or extract or both and D-mannose, and at least one component selected from the group consisting of ascorbic acid, bromelain and inulin, as well as effective or useful amounts of optional, but nevertheless useful components as herein described including suitable and pharmaceutically acceptable excipients, to the mammal in an effective amount for a time sufficient to prevent, reduce or eliminate symptoms associated with a urinary tract infection.

Suitable compositions useful for the methods of the present invention include combinations of the “primary” components, cranberry concentrate or extract or both, and D-mannose with at least one of the additional components, ascorbic acid, bromelain and inulin. Alternative combinations and permutations of these ingredients in useful compositions comprise effective amounts of the ingredients as set forth in the following table:

Alternative Compositional Embodiments

| Component | 1 | 2 | 3 | 4 | 5 | 6 |
|---|---|---|---|---|---|---|
| cranberry concentrate, | + | + | + | + | + | + |
| cranberry extract or both | | | | | | |
| D-mannose | + | + | + | + | + | + |
| ascorbic acid | + | + | + | − | − | − |
| bromelain | − | + | − | + | + | − |
| inulin | − | − | + | − | + | + |

+ = indicated component is present in the composition
− = indicated component is not present in the composition

It is believed that treatment in accordance with the present invention renders bacteria non-pathogenic and/or unable to colonize the urinary tract in an amount sufficient to cause symptoms of urinary tract infection and/or facilitates flushing of such bacteria from the urinary tract. Thus, one measure of efficacy includes monitoring the reduction or elimination of urinary bacterial counts associated with such infections during or after the course of treatment.

The term “treatment” is used herein to mean that administration of a composition of the present invention mitigates or substantially reduces urinary tract infections in mammals, including humans. Thus, the term “treatment” includes: substantially preventing urinary tract infections from occurring in a mammal, particularly when the mammal is predisposed to acquiring a urinary tract infection, but has not yet been diagnosed with the disease, inhibiting the urinary tract infection; and/or alleviating or reversing the urinary tract infection, especially substantially or completely.

Insofar as the methods of the present invention are directed to preventing a urinary tract infection, it is understood that the term “prevent” does not require completely keeping the urinary tract infection from occurring. Rather, as used herein, the term “preventing” refers to the ability of the skilled artisan to identify a person or population susceptible to urinary tract infections, such that administration of the compositions of the present invention can be made prior to the onset of the symptoms of the urinary tract infections. Populations that are at risk for a urinary tract infection include those who are subjected to: an obstruction of the bladder or urethra with resultant stasis of urine, insertion of instruments into the urinary tract (such as catheterization or cystoscopy), pregnancy, diabetes, and a history of analgesic nephropathy or reflux nephropathy. Furthermore, and significantly, the elderly population, especially those in long term care facilities, is at increased risk for developing UTIs due to lack of mobility and/or incomplete emptying of the bladder associated with conditions such as benign prostate hyperplasia, prostatitis, and urethral strictures, as well as incontinence involving the bowel and the bacteria normally found therein.

Thus, an at-risk patient population is identifiable and such a population can receive a composition of the present invention before initiation or substantial progression of the disease. Thus, for purposes of the present invention, urinary tract infections in such individuals or populations would be “prevented.”

Not only are compositions of the present invention suitable for prevention or treatment of urinary tract infections, but it has been unexpectedly found that at-risk individuals, such as those in long term nursing care facilities or persons with limited mobility, have experienced fewer falls when a composition of the present invention is administered. Without wishing to be bound by theory, such reduction in the number of falls may be a result of a reduced “urgency” associated with urination when a urinary tract infection is not present or with an improved mental status in an infection-free individual. In any event, the improvement has been observed and is believed to be associated with administration of compositions of the present invention. The reduction in fall rate for elderly persons in long term care facilities is particularly noteworthy, with the average number of falls prior to the use of compositions of the present invention at about 4 or more per month compared to fewer than about 2 falls per month during use of the compositions. Naturally, falls by elderly individuals will depend on the overall physical condition of the individual, but reductions in the number of falls of 50% or more have been observed during studies of the present compositions, including reductions of 60%, 70%, 80%, 90% or more.

The compositions of the present invention are also useful for prophylactic or acute treatment. Thus the compositions of the present invention can be administered in any way the skilled artisan in the field of medicine, pharmacology or nutrition would deem appropriate. It is apparent to the skilled artisan that preferred routes of administration can depend on factors including the stage of the urinary tract infection being treated or administered to for UTI prevention and the dosage form chosen. The oral route of administration is preferred.

The specific dose of a composition of the present invention to be administered, and the duration of treatment are typically mutually dependent. The dosage and treatment regimen will also depend upon such factors as the route of administration, the type of dosage form used, the infectious agent present, the ability of the combination to reach and sustain effective levels at the site of infection, the nature and extent of other infections (if any), the personal attributes of the subject (such as age and weight), the ability and/or willingness to comply with the treatment regimen, and the presence and severity of any side effects of the treatment.

For oral administration, compositions of the present invention, comprising at least the primary components, namely cranberry concentrate or extract or both and D-mannose, and at least one component selected from the group consisting of ascorbic acid, bromelain and inulin; for example, a particularly preferred composition comprising each of these recited components, typically for a human adult (weighing approximately 70 kilograms), are typically about 1000 mg to about 10,000 mg, preferably about 1500 mg to about 9500 mg, for example, about 4500 mg to about 5000 mg of the composition administered per day.

The foregoing notwithstanding, it is to be understood that dosages or amounts set forth herein are exemplary only and they are not intended to, and do not to any extent, limit the scope of practice of the present invention, particularly by one skilled in the art, including a physician, a pharmacist, a registered or practical nurse, a nutritionist and the like.

Compositions of the present invention, including compositions having a volume of about 30 cc or 1 ounce as described herein, can be administered at least once daily, for example, from about once to about five times daily including at a frequency of twice, three times and four times daily. Administration of about 30 cc of a preferred composition of the invention twice daily is particularly preferred. Preferably, compositions can be administered on a continuing basis, particularly for prevention of urinary tract infections, such as for at least about once weekly, twice weekly, three times weekly, four times weekly, and five times weekly. Alternatively, compositions can be administered daily or twice or more times daily, depending on the volume of each composition ingested or administered. Treatment of an existing infection may dictate administration of the inventive composition on a more frequent basis until the symptoms of the infection are ameliorated and then continuing administration on a continuing, but less frequent basis as a preventative. It is understood that the expressed dosages and administration frequencies are by way of example only, and that administration can be adjusted depending on the individual physical characteristics typically within the abilities of person skilled in the art, as noted above.

A further embodiment of the present invention includes an article of manufacture or kit comprising a container comprising: (I) a liquid concentrate mixture having a volume of about one ounce or about 30 ml comprising cranberry concentrate or extract or both, and D-mannose and at least one component selected from the group consisting of ascorbic acid, bromelain and inulin. A particularly preferred article or kit comprises a liquid concentrate (I) comprising about 2000 mg of a 4:1 cranberry concentrate or a suitable amount of cranberry extract; about 250 mg of D-mannose; about 375 mg ascorbic acid; about 2000 mcu bromelain; and about 250 mg inulin; and (II) instructions for administering the concentrate on a periodic basis. The composition included in the article of manufacture optionally further includes additional components as described hereinabove and the instructions for administering the concentrate composition can include alternative administration intervals from one or more of those described above.

In another embodiment comprising a kit or article of manufacture, the primary components and optional components can be provided in a unit dosage form, cumulatively comprising a suitable amount of the combination, with instructions for adding a suitable amount of filtered water or other suitable liquid in order to prepare a dosage for immediate use or for consumption as convenient, consistent with stability of the components of the combination, e.g., selected from the group consisting of within about 2 hours, about 4 hours, about 8 hours, about 16 hours and about 24 hours. A suitable time period can be extended or shortened, as one skilled in the art would recognize based on the availability of a temperature controlled environment, such as a refrigerator, for storing a prepared liquid mixture so as to prevent undesirable degradation of the components, including primary as well as optional components. Alternatively, the amount of the composition exceeds a single dose and the kit further includes appropriate instructions as well as a means for measuring an appropriate amount, e.g., by weight or volume, in order to prepare a single dose by admixing a suitable amount of the composition with a liquid or other ingestible component for administration to the patient.

### EXAMPLES

### Example 1

A mixture is prepared of the following primary and optional components: (I) Primary: about 2000 mg of a 4:1 cranberry concentrate; about 250 mg of D-mannose; about 375 mg ascorbic acid; about 2000 milk clotting units (mcu) bromelain; and about 250 mg inulin; and (II) Optional: water, glycerin, phosphoric acid, sodium benzoate, potassium sorbate, sucralose, acesulfame K, and natural color. The total volume of the mixture is about one ounce or 30 ml. The resulting composition is suitable for use in preventing or treating urinary tract infections in humans, and is administered or provided on a periodic basis, such as at least once daily, preferably twice daily. Further benefits are obtained when total fluid intake is managed so as to maintain proper hydration, such as about 1500 ml to about 1600 ml total fluid intake daily.

### Example 2

A multistate, multicenter interventional prospective trial was conducted in long term care (LTC) facilities in 5 different states involving 124 elderly residents of the LTC facilities. The participants included male and female subjects from 10 LTC sites. Asymptomatic residents with 2 or more UTIs in the past 12 months were included in the study, as were those who exhibited changes in mental status/confusion consistent with UTI prodromes. Exclusion criteria included administration of antibiotics within 48 hours prior to the study or long-term treatment with antibiotics for chronic UTI; stones in urinary tract; allergy to cranberry compounds; current use of warfarin; major cardiac or renal problem; symptomatic UTI (>3 symptoms based on the McGeer criteria, described below); fluid restriction; and thickened liquid. The interventional trial was conducted in accordance with the Good Clinical Practice regulations of the Food and Drug Administration. Trained personnel were available on-site to facilitate adherence to the study protocol. Written informed consent was obtained from participants or their legally appointed representatives.

Each participant received 30 ml (1 oz.) of a composition of the present invention, referred to as a cranberry concentrate complex, administered twice daily, combined with a goal of 1500 cc to 1600 cc of fluid intake; the study lasted for 90 days. The natural cranberry concentrate complex liquid (UTI-STAT) was provided by Medical Nutrition USA, Inc. (Englewood, N.J.). Each 30 ml (1 oz.) contained a blend of cranberry 1.5% alpha proanthocyanidin concentrate, dextro-mannose, ascorbic acid, bromelain, and prebiotic fructo-oligosaccharides (FOS), corresponding to a composition comprising: a liquid concentrate mixture having a volume of about one ounce or about 30 ml comprising about 2000 mg of a 4:1 cranberry concentrate; about 250 mg of D-mannose; about 375 mg ascorbic acid; about 2000 milk clotting units (mcu) bromelain; about 250 mg inulin; and further comprising water, glycerin, phosphoric acid, sodium benzoate, potassium sorbate, sucralose, acesulfame K, and natural color.

A total of 88 residents completed the three-month study; of these, 5 were male. Ten percent (12/124) withdrew because of flu; 100 (11/124) were transferred to hospitals; there was one death (1/124); and 10% (12/124) withdrew for other reasons. A total of 72 of 88 (80%) of the residents in the study exceeded the fluid intake target of >1500 cc/d (based on 30 cc/kg of body weight adjusted for cardiac and renal status); 10% (12/8) averaged 1180 to 1475 cc/d and 5% (4/88) averaged 939 c/d.

All outcomes were assessed by Directors of Nursing (DONs) and Associate Directors of Nursing (ADONs). The primary outcome was prevention of UTI symptoms. These were defined as urinary pain, urinary burning, difficulty urinating, frequent urination, urinary urgency, urine with odor, cloudy urine, dark urine, blood in urine, chills/fever, abdominal pain, mental status change, or falls. 81 out of 88 (92%) remained symptom-free, in other words, no symptoms of urinary tract infection for the full three months of the study and thus they did not require antibiotic therapy. A total of 2 of residents had mild urinary pain for up to 5 weeks; 2 had mild urinary burning for up to 6 weeks; 2 had mild urinary odor for 4 weeks; and 1 had dark urine for 6 weeks; each of these 7 individuals had <3 symptoms, and, in consultation with a physician, did not meet McGeer criteria for the diagnosis of a UTI (McGeer A. et al., Definitions of infection for surveillance in long-term care facilities. Am J Infect Control. 1991; 19:1-7. The McGeer criteria specify that 3 of the following symptoms must be present for the diagnosis of a UTI: fever ≧38° C. (100° F.); new or increased frequency, urgency, or burning on urination; new flank or suprapubic pain or tenderness; change in character of urine; or worsening of mental or functional status). All other residents taking the composition remained comfortable and asymptomatic. All sites facilities) achieved compliance with CMS (Centers for Medicare and Medicaid Services) Nursing Home Quality Improvement measures (NHQI) of a 30% prophylaxis rate and a rate of UTIs below the 2007 national level of 9.9%.

Additionally, none of the residents experienced falls during the study whereas all had one or more fall prior to the study. It was concluded that the study supported the efficacy of a natural cranberry concentrate complex liquid combined with increased hydration in preventing recurrence of UTI symptoms in residents of LTC facilities.

This study was the first of its kind to conclusively show the benefits of a cranberry concentrated complex liquid, specifically a composition of the present invention, in improving quality of life indicator symptoms in LTC residents at risk for urinary tract infections. The study further supports the efficacy of such compositions combined with increased hydration in preventing recurrence of UTI symptoms in residents of LTC facilities.

For purposes of the present invention the following terms shall have the indicated meaning:

Comprise or comprising: Throughout the entire specification, including the claims, the word “comprise” and variations of the word, such as “comprising” and “comprises,” as well as “have,” “having,” “includes,” “include” and “including,” and variations thereof, means that the named steps, elements or materials to which it refers are essential, but other steps, elements or materials may be added and still form a construct with the scope of the claim or disclosure. When recited in describing the invention and in a claim, it means that the invention and what is claimed is considered to be what follows and potentially more. These terms, particularly when applied to claims, are inclusive or open-ended and do not exclude additional, unrecited elements or methods steps.

Consisting essentially of: In the present context, “consisting essentially of” is meant to exclude any element or combination of elements as well as any amount of any element or combination of elements that would alter the basic and novel characteristics of the invention. Thus, by way of example and not limitation, prevention or treatment of urinary tract infection that does not include the use of an edible liquid concentrate with at least cranberry concentrate and inulin would be excluded.

Substantially: Unless otherwise defined with respect to a specific property, characteristic or variable, the term “substantially” as applied to any criteria, such as a property, characteristic or variable, means to meet the stated criteria in such measure such that one skilled in the art would understand that the benefit to be achieved, or the condition or property value desired is met.

Any range of numbers recited in the specification hereinabove or in the paragraphs and claims hereinafter, referring to various aspects of the invention, such as that representing a particular set of properties, units of measure, conditions, physical states or percentages, is intended to literally incorporate expressly herein by reference or otherwise, any number falling within such range, including any subset of numbers or ranges subsumed within any range so recited. For example, whenever a numerical range with a lower limit, R_{L}, and an upper limit R_{U}, is disclosed, any number R falling within the range is specifically disclosed. In particular, the following numbers R within the range are specifically disclosed: R=R_{L}+k(R_{U}−R_{L}), where k is a variable ranging from 1% to 100% with a 1% increment, e.g., k is 1%, 2%, 3%, 4%, 5% . . . 50%, 51%, 52% . . . 95%, 96%, 97%, 98%, 99%, or 100%. Moreover, any numerical range represented by any two values of R, as calculated above is also specifically disclosed.

Furthermore, the term “about” when used as a modifier for, or in conjunction with, a variable, characteristic or condition is intended to convey that the numbers, ranges, characteristics and conditions disclosed herein are flexible and that practice of the present invention by those skilled in the art using times, concentrations, amounts, contents, properties such as size, weight, surface area, etc., that are outside of the range or different from a single value, will achieve the desired results as described in the application, namely, compositions comprising a combination of components suitable for preventing or treating urinary tract infections in mammals, the compositions comprising: cranberry concentrate, cranberry extract or both; D-mannose; ascorbic acid; bromelain; and inulin.

All documents described herein are incorporated by reference herein, including any patent applications and/or testing procedures. The principles, preferred embodiments, and modes of operation of the present invention have been described in the foregoing specification.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the various aspects or embodiments of the present invention as set forth in the application and the appended claims.

1. A composition comprising a combination of components suitable for preventing or treating urinary tract infections in mammals, including humans, the composition comprising an effective amount of:
(A) cranberry concentrate, cranberry extract or both;
(B) D-mannose; and
at least one component selected from the group consisting of:
(C) ascorbic acid;
(D) bromelain; and
(E) inulin.

2. The composition of claim 1 wherein the composition is in liquid form.

3. The composition of claim 1 wherein cranberry concentrate is prepared from a member selected from the group consisting of cranberry fruits, cranberry juice, cranberry juice extract, cranberry juice powder, cranberry powder, cranberry powder extract and combinations thereof.

4. The composition of claim 1 wherein cranberry concentrate is a 4:1 concentrate and is present in an amount of from about 400 mg to about 3000 mg.

5. The composition of claim 1 or claim 4 wherein D-mannose is present in an amount of from about 100 mg to about 1000 mg.

6. The composition of claim 1 or claim 4 wherein D-mannose is present in an amount of from about 100 mg to about 1000 mg.

7. The composition of claim 1 wherein ascorbic acid is present in an amount of from about 50 mg to about 1000 mg.

8. The composition of claim 1 or claim 7 wherein bromelain is present in an amount of from about 500 milk clotting units (mcu) to about 3000 mcu.

9. The composition of claim 1 or claim 7 wherein inulin is present in an amount of from about 100 mg to about 15000 mg.

10. The composition of claim 1 selected from the group consisting of admixtures 1, 2, 3, 4, 5, and 6 according to the following table:
Admixture

| Component | 1 | 2 | 3 | 4 | 5 | 6 |
|---|---|---|---|---|---|---|
| cranberry | + | + | + | + | + | + |
| concentrate, | | | | | | |
| cranberry extract | | | | | | |
| or both | | | | | | |
| D-mannose | + | + | + | + | + | + |
| ascorbic acid | + | + | + | − | − | − |
| bromelain | − | + | − | + | + | − |
| inulin | − | − | + | − | + | + |

wherein + indicates the component is present in the composition; and
wherein − = indicates the component is not present in the composition.

11. The composition of claim 1 further comprising at least one component selected from the group consisting of water, glycerin, phosphoric acid, sodium benzoate, potassium sorbate, sweetener, natural fruit concentrate other than cranberry, flavoring agent, coloring agent and mixtures thereof.

12. The composition of claim 11 wherein the sweetener is selected from the group consisting of sugar, sucralose, acesulfame K, aspartame, saccharin and mixtures thereof.

13. The composition of claim 2 in a total volume in ounces selected from the group consisting of about 0.5 to about 1.5; about 0.75 to about 1.25; and about 0.9 to about 1.1.

14. The composition of claim 13, each 30 ml comprising about 2000 mg of a 4:1 cranberry concentrate; about 250 mg of D-mannose; about 375 mg ascorbic acid; about 2000 mcu bromelain; and about 250 mg inulin.

15. The composition of claim 1 wherein the composition is in a form selected from the group consisting of solid, particulate, powdered form and mixtures thereof.

16. The composition of claim 2 having a liquid volume of about one ounce or about 30 ml and comprising about 2000 mg of a 4:1 cranberry concentrate; about 250 mg of D-mannose; about 375 mg ascorbic acid; about 2000 mcu bromelain; and about 250 mg inulin.

17. A composition suitable for preventing or treating urinary tract infections in humans, the composition comprising: a liquid concentrate mixture having a volume of about one ounce or about 30 ml comprising about 2000 mg of a 4:1 cranberry concentrate; about 250 mg of D-mannose; about 375 mg ascorbic acid; about 2000 milk clotting units (mcu) bromelain; about 250 mg inulin; and further comprising water, glycerin, phosphoric acid, sodium benzoate, potassium sorbate, sucralose, acesulfame K, and natural color.

18. A method for preventing or treating urinary tract infections in mammals, comprising administering to the mammal a composition of claim 1, claim 2 or claim 10 to prevent, reduce or eliminate symptoms associated with the infection.

19. The method of claim 18, wherein the composition is in the form of a liquid concentrate in an amount of about one ounce.

20. The method of claim 19 wherein the composition is administered to the mammal on a frequency selected from the group consisting of a regular, continuing and an on-going basis.

21. The method of claim 20 wherein the composition is administered at a frequency selected from the group consisting of at least once weekly, at least twice weekly, at least three times weekly, at least four times weekly, at least five times weekly and daily.

22. The method according to claim 18 wherein the composition the mammal further ingests on a daily basis about 900 ml to about 1750 ml of fluids.

23. The method according to claim 18 wherein the mammal is a human and further including a reduction in the frequency of falls experienced by the human.

24. A method for making a composition suitable for use in preventing or treating urinary tract infections in mammals, including humans, comprising:
providing an effective amount of:
(A) cranberry concentrate, cranberry extract or both;
(B) D-mannose; and
at least one component selected from the group consisting of:
(C) ascorbic acid;
(D) bromelain; and
(E) inulin;
mixing an effective amount of each component with one another and including sufficient water in order to produce a liquid concentrate mixture of about 0.5 ounces to about 1.5 ounces in volume.

25. The method of claim 24, wherein a component, when present, is present in the following amount:
cranberry concentrate in the form of a 4:1 concentrate in an amount of from about 400 mg to about 3000 mg; D-mannose in an amount of from about 100 mg to about 1000 mg; ascorbic acid in an amount of from about 50 mg to about 1000 mg; bromelain in an amount of from about 500 milk clotting units (mcu) to about 3000 mcu; and inulin in an amount of from about 100 mg to about 15000 mg.

26. The method of claim 25 wherein the mixture further comprises a component selected from the group consisting of water, glycerin, phosphoric acid, sodium benzoate, potassium sorbate, sweetener, natural fruit concentrate other than cranberry, flavoring agent, coloring agent and mixtures thereof.

27. An article of manufacture comprising a container having (I) a composition according to claim 1 or claim 10; and (II) instructions for administering the concentrate on a periodic basis.

28. The article of manufacture according to claim 27 comprising: (I) a liquid concentrate mixture having a volume of about one ounce or about 30 ml comprising about 2000 mg of a 4:1 cranberry concentrate; about 250 mg of D-mannose; about 375 mg ascorbic acid; about 2000 mcu bromelain; and about 250 mg inulin.
